(19) United States
(12) Patent Application Publication
(10) Pub. No.: US 2026/0330946 A1
(43) Pub. Date: Oct. 22, 2026

(54) SELF-CLEANING DETECTOR WITH TRANSMISSION

(71) Applicant: Microchip Technology Inc., Chandler, AZ (US)

(72) Inventors: Patrick McFarland, Gilbert, AZ (US); Arthur B. Eck, Gilbert, AZ (US); Bomy Chen, Newark, CA (US); Steve Nagel, Chandler, AZ (US)

(73) Assignee: Microchip Technology Inc., Chandler, AZ (US)

(21) Appl. No.: 19/245,298

(22) Filed: Jun. 21, 2025

Related U.S. Application Data
(60) Provisional application No. 63/791,784, filed on Apr. 21, 2025.

(51) Int. Cl. G08B 17/10; B08B 7/02; G01D 3/036; G01N 33/00; H04R 17/10

(52) U.S. Cl. CPC G08B 17/10; G01N 33/004; G01N 33/0075; H04R 17/10; B08B 7/028; G01D 3/036

(57) ABSTRACT

A monitoring system is disclosed. The monitoring system includes a sensor and a piezoelectric speaker. The monitoring system also includes a control circuit. The control circuit is configured to drive the piezoelectric speaker to produce acoustic waves within a human-audible frequency range in response to the sensor. The control circuit is also configured to drive the piezoelectric speaker to produce ultrasonic acoustic waves that simultaneously vibrate the sensor and convey a monitoring-network signal.

### PRIORITY

This application claims priority to commonly owned U.S. Provisional Patent Application No. 63/791,784 filed Apr. 21, 2025, the entire contents of which are hereby incorporated by reference for all purposes.

### TECHNICAL FIELD

The present disclosure relates to electronic devices, including monitoring systems and sensors. Various examples of the teachings herein include systems and methods for energy-efficient self-cleaning monitoring systems and environmental-safety sensors (for example, smoke detectors, carbon monoxide detectors, among others).

### BACKGROUND

Monitoring systems, such as carbon monoxide and smoke detectors, have become a standard safety feature in residential and commercial buildings, playing a crucial role in early fire and carbon monoxide detection and prevention of loss of life and property damage. Other forms of monitoring systems or life-safety sensors that may be used in either residential, commercial, or industrial buildings, may detect the presence of other harmful natural gasses such as radon, or detect the presence of various environmental conditions such as humidity.

In the field of monitoring systems, a build-up of dust and debris may adversely affect the operation of said devices. For example, such build-up of dust or debris may reduce the accuracy of sensor readings and the effectiveness of the monitoring system. Failure to reduce or remove such contamination may reduce the accuracy and lifespan of the sensor and possibly the entire monitoring system. Thus, the build-up of dust or debris may cause the operation of monitoring systems, such as smoke detectors and other life-safety monitors, to be compromised beyond acceptable safety limits.

Cleaning monitoring systems by hand may be time-consuming or otherwise complicated. For example, in some monitoring systems, cleaning requires accessing an interior chamber of the monitoring system. Various self-cleaning devices may incorporate mechanical means such as air movement to remove some forms of contamination. Such self-cleaning may consume power, thereby reducing energy efficiency of the monitoring system.

### SUMMARY

The examples herein describe monitoring systems and methods for monitoring safety hazards (such as the presence of smoke, carbon monoxide, or other harmful gasses) or environmental conditions (such as humidity level) while also providing for the simultaneous conveyance of network-communication signals and self-cleaning of the monitoring system.

According to one example, a monitoring system includes a sensor, a piezoelectric speaker, and a control circuit configured to drive the piezoelectric speaker to produce ultrasonic acoustic waves that simultaneously vibrate the sensor and convey a monitoring-network signal. In some examples, the monitoring-network signal indicates at least one of a status of the monitoring system, a detected hazard, and a detected environmental condition. In the same or different examples, the ultrasonic acoustic waves are in a frequency range greater than 20 kHz and less than or equal to 250 kHz. In the same or different examples, the control circuit is further configured to drive the piezoelectric speaker to produce acoustic waves within a human-audible frequency range in response to the sensor. In the same or different examples, the piezoelectric speaker has a resonant frequency within the human-audible frequency range. In the same or different examples, the piezoelectric speaker is directionally aimed at the sensor. In the same or different examples, the monitoring system further includes a microphone configured to receive a second monitoring-network signal via second ultrasonic acoustic waves. In the same or different examples, the control circuit is configured to drive the piezoelectric speaker to rebroadcast the second monitoring-network signal received by the microphone. In the same or different examples, the control circuit comprises a modulator configured to modulate the monitoring-network signal onto a carrier wave. In the same or different examples, the modulator performs frequency modulation. In the same or different examples, the monitoring-network signal includes a digitally-encoded address identifying the monitoring system. In the same or different examples, the sensor is configured to detect at least one of smoke and carbon monoxide.

According to another example, a monitoring network includes a plurality of monitoring systems configured to communicate ultrasonically, wherein each monitoring system includes a sensor, a piezoelectric speaker, and a control circuit configured to drive the piezoelectric speaker to produce ultrasonic acoustic waves that simultaneously vibrate the sensor and convey a monitoring-network signal. In some examples, each monitoring system further includes a microphone configured to receive a second monitoring-network signal from another of the plurality of monitoring systems. In the same or different examples, the control circuit of each monitoring system is configured to drive the piezoelectric speaker to rebroadcast the second monitoring-network signal. In the same or different examples, the monitoring network further comprises a listening station including one or more microphones to receive the monitoring-network signal. In the same or different examples, the listening station is communicatively coupled to a central panel and configured to message the central panel in response to receiving the monitoring-network signal from one of the plurality of monitoring systems. In the same or different examples, the respective control circuits of two or more of the plurality of monitoring systems are configured to perform a frequency sweep across multiple ultrasonic carrier frequencies to test a signal strength of communications therebetween at the multiple ultrasonic carrier frequencies, and to select an ultrasonic carrier frequency for a transmission of the monitoring-network signal.

Another example provides a method for operating a monitoring system including detecting with a sensor at least one of a hazard and an environmental condition, driving a piezoelectric speaker to produce acoustic waves within a human-audible frequency range in response to the sensor, and driving the piezoelectric speaker to produce ultrasonic acoustic waves to simultaneously vibrate the sensor and convey a monitoring-network signal. In some examples, the piezoelectric speaker is directionally aimed at the sensor. In the same or different examples, the method further comprises modulating the monitoring-network signal onto a carrier wave. In the same or different examples, the method further comprises receiving a second monitoring-network signal via second ultrasonic acoustic waves, and rebroadcasting the second monitoring-network signal.

### BRIEF DESCRIPTION OF THE DRAWINGS

A more complete understanding may be acquired by referring to the following description taken in conjunction with the accompanying drawings, in which like reference numbers indicate like features.

FIG. 1 illustrates a monitoring system in accordance with various examples of the present disclosure.

FIG. 2A illustrates a monitoring system in accordance with various examples of the present disclosure.

FIG. 2B illustrates a schematic block diagram of a monitoring system in accordance with various examples of the present disclosure.

FIG. 3 illustrates a monitoring network in accordance with various examples of the present disclosure.

FIG. 4 illustrates a monitoring network in accordance with various examples of the present disclosure.

FIG. 5 illustrates a flow chart of an example method for operating a monitoring system in accordance with various examples of the present disclosure.

FIG. 6 illustrates a flow chart of an example method for operating a monitoring system in accordance with various examples of the present disclosure.

FIG. 7 illustrates a flow chart of an example method for operating a monitoring system in accordance with various examples of the present disclosure.

### DETAILED DESCRIPTION

The following description sets forth example aspects of the present disclosure. Such description is not intended as a limitation on the scope of the present disclosure. Rather, the description also encompasses combinations and modifications to those example aspects described herein.

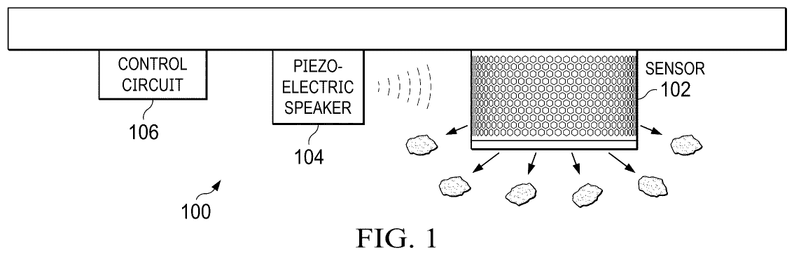

FIG. 1 illustrates monitoring system 100 in accordance with various examples of the present disclosure. Monitoring system 100 may be implemented in any suitable fashion according to the operation described in the present disclosure. In various examples, monitoring system 100 may include sensor 102, piezoelectric speaker 104, and control circuit 106. In some examples, sensor 102 may be configured to detect at least one of smoke and carbon monoxide. In addition, or in the alternative, sensor 102 may be configured to detect other harmful gasses, including but not limited to, radon for example. In addition, or in the alternative, sensor 102 may be configured to detect one or more environmental conditions such as humidity level. In response to detecting a hazard (such as the presence of smoke, carbon monoxide, or other hazardous gas) or an environmental condition (such as humidity level), sensor 102 may communicate the detected hazard or environmental condition to control circuit 106.

Control circuit 106 may be implemented in any suitable fashion according to the operation described in the present disclosure. For example, control circuit 106 may be configured to receive a signal from sensor 102 indicating a detected hazard or other environmental condition. In response to sensor 102, control circuit 106 may initiate an audible alert. For example, control circuit 106 may be configured to drive piezoelectric speaker 104 to produce acoustic waves within a human-audible frequency range in response to the sensor 102, and in particular in response to a signal from sensor 102 indicating the presence of a hazard or other environmental condition. For the purposes of the present disclosure, the human-audible frequency range may refer to frequencies within the range of 20 Hz to 20 kHz. In various examples, piezoelectric speaker 104 may have a resonant frequency within the human-audible frequency range. For example, piezoelectric speaker 104 may be configured to have a resonant frequency at or around 3.1 kHz. By configuring piezoelectric speaker 104 with a resonant frequency in the human-audible frequency range, the volume of the audible alert produced by piezoelectric speaker 104 in response to a detected hazard or environmental condition may be increased as compared to otherwise similar systems where the resonant frequency of the piezoelectric speaker is outside the human-audible frequency range.

In various examples, control circuit 106 may also be configured to drive piezoelectric speaker 104 to produce ultrasonic acoustic waves (waves outside of the human-audible frequency range). For example, the ultrasonic acoustic waves may be in a frequency range greater than 20 kHz and less than or equal to 250 kHz. Such ultrasonic acoustic waves may be directed toward sensor 102, and may thereby deliver acoustic vibrations to sensor 102. For example, piezoelectric speaker 104 may be directionally aimed at sensor 102 specifically to increase the strength of the acoustic vibrations caused at sensor 102.

As described in further detail below, such acoustic vibrations may dislodge dust or debris from sensor 102, thereby preventing a build-up of dust or debris that would otherwise negatively impact the operation of monitoring system 100. For example, as shown in FIG. 1, sensor 102 may include a guard, such as an insect mesh, which may prevent insects, dust, or other debris from contaminating one or more sensing elements located within the chamber defined by the guard. In various examples, the guard may define the test chamber for monitoring system 100. By projecting ultrasonic acoustic waves toward sensor 102, the resulting acoustic vibrations may cause any insects, dust, or other debris, to be dislodged from the protective guard and from other portions of sensor 102. Once dislodged, the force of gravity may further operate to remove such contaminants from sensor 102 and from monitoring system 100 as a whole. Thus, monitoring system 100 may prevent unwanted blockage of air flow to the internal sensing elements of sensor 102.

In various examples, control circuit 106 may be configured to drive piezoelectric speaker 104 to produce ultrasonic acoustic waves to self-clean sensor 102 on a periodic basis. The use of ultrasonic acoustic waves may allow for such self-cleaning while avoiding the creation of a human-audible noise that may annoy or falsely alarm a user.

In various examples, multiple instances of a monitoring system, such as monitoring system 100, may be included within a monitoring network. For example, in a residential or commercial building, different rooms or areas may each include a monitoring system such as monitoring system 100. Individual instances of monitoring system 100 may communicate with each other or with a central listening station or central panel. For example, individual instances of monitoring system 100 may communicate a system status, such as an operating status or a low-battery level, among other status updates, to other monitoring systems (such as other instances of monitoring system 100) or to a central listening station or central panel. Individual instances of monitoring system 100 may also communicate a detected hazard or environmental condition to other monitoring systems (such as other instances of monitoring system 100) or to a central listening station or central panel. Thus, when one monitoring system 100 detects a hazard, the presence of that hazard can be communicated to other monitoring systems in the building, which may then collectively provide an all-room warning (such as an audible alert in each room) to building occupants.

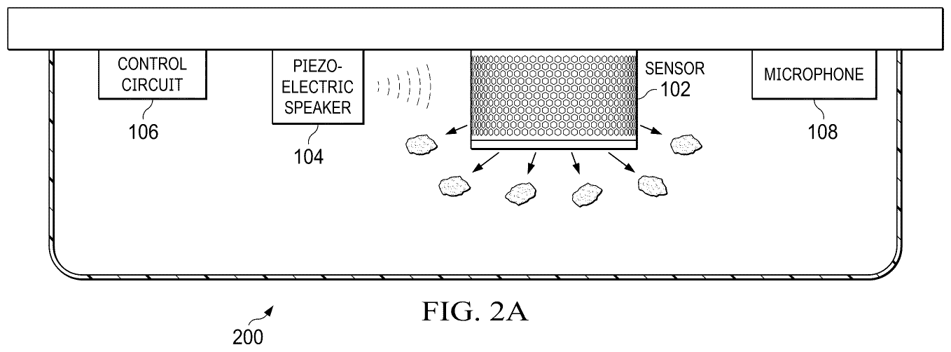

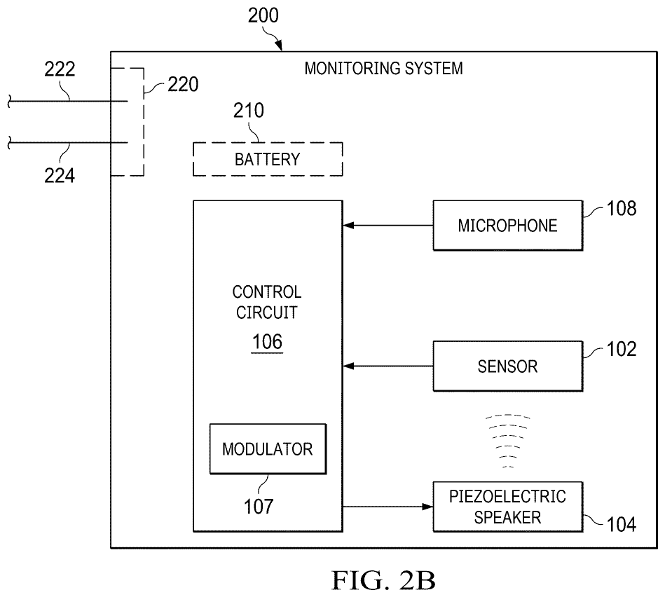

In various examples, control circuit 106 may be configured to drive piezoelectric speaker 104 to produce ultrasonic acoustic waves that simultaneously vibrate sensor 102 and convey a monitoring-network signal. The monitoring-network signal may indicate at least one of a status of monitoring system 100, a detected hazard, and a detected environmental condition. For example, control circuit 106 may include a modulator (such as modulator 107 shown in FIG. 2B) that may modulate sensor data, such as a sensor status or a detected hazard or environmental condition, into a carrier frequency that is used for the self-cleaning of sensor 102. Control circuit 106 may drive piezoelectric speaker 104 with the modulated signal to simultaneously provide for both self-cleaning of sensor 102 and for network communication.

FIG. 2A illustrates monitoring system 200 in accordance with various examples of the present disclosure. Monitoring system 200 may be implemented in any suitable fashion according to the operation described in the present disclosure. In some examples, monitoring system 200 may include sensor 102, piezoelectric speaker 104, and control circuit 106, which may operate in a similar manner as described above for FIG. 1. As shown in FIG. 2A, monitoring system 200 may further include microphone 108.

Microphone 108 may be configured to receive a second monitoring-network signal via second ultrasonic acoustic waves. For example, in addition to the control circuit 106 driving piezoelectric speaker 104 to produce ultrasonic acoustic waves that simultaneously vibrate sensor 102 and convey a monitoring-network signal, microphone 108 may receive a second monitoring-network signal via second ultrasonic acoustic waves from another instance of monitoring system 200 (or from an instance of monitoring system 100).

In various examples, monitoring system 200 may serve as a node in a mesh monitoring-network system. For example, one or more instances of monitoring system 200 may mesh together to allow for the rebroadcasting of network signals from other instances of monitoring system 200. To facilitate such operation, control circuit 106 may be configured to drive piezoelectric speaker 104 to rebroadcast the second monitoring-network signal received by microphone 108. In various examples, monitoring system 200 may be tuned to rebroadcast depending on the signal strength of the received signal. For example, if the second monitoring-networks signal received by microphone 108 from a different instance of monitoring system 200 (or other monitoring system) is below a signal-strength threshold, control circuit 106 may drive piezoelectric speaker 104 to rebroadcast the second monitoring-network signal. Accordingly, monitoring-network signals sent by an instance of monitoring system 200 may traverse across the mesh network to be received, for example, by a central listening station or central panel that may have otherwise been out of range of one or more of the instances of monitoring system 200.

As described above with reference to FIG. 1, control circuit 106 may be configured to drive piezoelectric speaker 104 to produce ultrasonic acoustic waves that simultaneously vibrate sensor 102 and convey a monitoring-network signal. The monitoring-network signal may indicate at least one of a status of monitoring system 200, a detected hazard, and a detected environmental condition. For example, control circuit 106 may include a modulator (such as modulator 107 shown in FIG. 2B) that may modulate sensor data, such as a sensor status or a detected hazard or environmental condition, into a carrier frequency that is used for the self-cleaning of sensor 102. Control circuit 106 may drive piezoelectric speaker 104 with the modulated signal to provide for both self-cleaning of sensor 102 and for communication. Further, as described above, control circuit 106 may be configured to drive piezoelectric speaker 104 to rebroadcast a second monitoring-network signal received by microphone 108 via second ultrasonic acoustic waves. For example, in response to receiving a second monitoring-network signal via microphone 108, control circuit 106 may drive piezoelectric speaker 104 with a modulated signal to reproduce the second ultrasonic acoustic waves and thereby provide for both self-cleaning of sensor 102 and for the rebroadcasting of the second monitoring-network signal.

Combining self-cleaning operations and network communication into the same transmission of ultrasonic acoustic waves, monitoring systems such as monitoring system 100 and monitoring system 200 may reduce costs and improve energy efficiency. For example, monitoring system 100 or monitoring system 200 may be battery powered devices. By combining the self-cleaning operations with the transmission of network communications, the number of times that control circuit 106 actuates piezoelectric speaker 104 may be reduced, thereby reducing power consumption. For example, where monitoring system 100 or monitoring system 200 are battery powered devices, such power savings may extend battery life. Further, reducing the number of times that control circuit 106 actuates piezoelectric speaker 104 to produce acoustic vibrations may reduce the collective physical strain of those vibrations on the monitoring system components.

Additionally, utilizing the ultrasonic acoustic waves for network communication in addition to self-cleaning of sensor 102 may reduce the cost of installation as well as the cost of the device. For example, in some panel-based monitoring networks, all monitoring systems included therein may be on the same power line and may use the same interconnect to communicate. By providing network communication via ultrasonic acoustic waves, the additional interconnect line may be omitted, thereby reducing the complexity and cost of installing a monitoring network including numerous monitoring systems (such as monitoring system 100 or monitoring system 200). Further, by providing network communication via ultrasonic acoustic waves, other hardware typically used for wireless communication (such as RF communication modules or Wi-Fi communication modules) may be omitted from monitoring system 100 and monitoring system 200. Such omission of RF or Wi-Fi communication modules may reduce the cost of the monitoring system and also further reduce power consumption.

FIG. 2B illustrates a schematic block diagram of monitoring system 200 in accordance with various examples of the present disclosure. As shown in FIG. 2B, monitoring system 200 may include sensor 102, piezoelectric speaker 104, control circuit 106, modulator 107, and microphone 108, which may operate as described above with reference to FIG. 2A. As also shown in FIG. 2B, monitoring system 200 may optionally include battery 210. Battery 210 may be configured to provide power to the internal components of monitoring system 200. In some examples, battery 210 may serve as a backup power supply and may supply power to the components of monitoring system 200 in the absence of power from a mains power line, for example in the event of a power outage. In such examples, battery 210 may be a rechargeable battery that charges when AC power is provided to monitoring system 200 as a whole. In other examples, battery 210 may be configured as the primary power source providing continuous power to the components of monitoring system 200.

As shown in FIG. 2B, monitoring system 200 may also optionally include wiring assembly 220. Wiring assembly 220 may include a power line 222 configured to provide power to the internal components of monitoring system 200. Wiring assembly 220 may also include ground line 224. Ground line 224 may provide an electrical return for an external power source that ultimately supplies the power for power line 222.

As described above with reference to FIG. 1 and FIG. 2A, by combining self-cleaning operations and network communication into the same transmission of ultrasonic acoustic waves, monitoring system 200 may reduce costs and improve energy efficiency. For example, in various examples, monitoring system 200 may be a battery-powered device, powered for example by battery 210. By combining the self-cleaning operations with the transmission of network communications, the number of times that control circuit 106 actuates piezoelectric speaker 104 may be reduced, thereby reducing power consumption from battery 210 and extending the life of battery 210.

Additionally, utilizing the ultrasonic acoustic waves for network communication in addition to self-cleaning of sensor 102 may reduce the cost of installation. For example, in some panel-based monitoring networks, all monitoring systems included therein may be on the same power line and may use the same interconnect to communicate. By providing network communication via ultrasonic acoustic waves, the additional interconnect line may be omitted. As shown in FIG. 2B, such an additional interconnect line may be omitted from wiring assembly 220, thereby reducing the complexity and cost of installing a monitoring network including numerous instances of monitoring system 200. Further, by providing network communication via ultrasonic acoustic waves, other hardware typically used for wireless communication (such as RF communication modules or Wi-Fi communication modules) may be omitted from monitoring system 200. Such omission of RF or Wi-Fi communication modules may reduce the cost of the monitoring system and also further reduce power consumption thereby extending the life of battery 210.

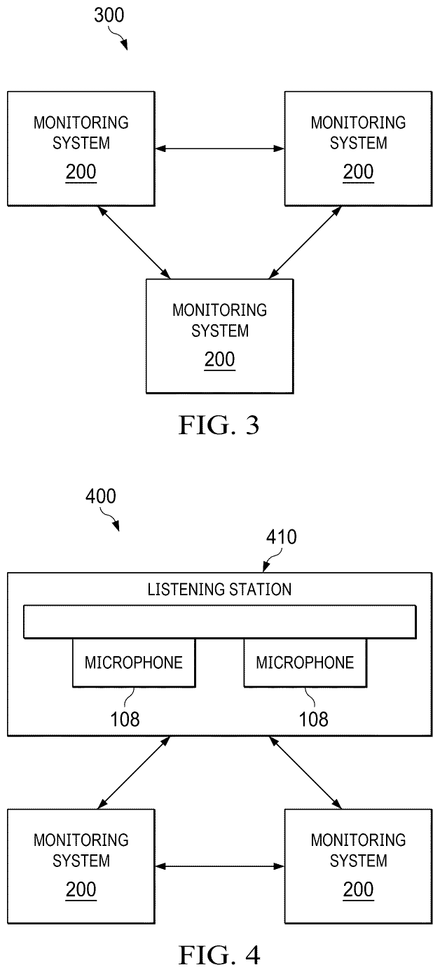

FIG. 3 illustrates monitoring network 300 in accordance with various examples of the present disclosure. Monitoring network 300 may be implemented in any suitable fashion according to the operation described in the present disclosure. As shown in FIG. 3, monitoring network 300 may include a plurality of monitoring systems, such as monitoring system 200. The plurality of monitoring systems may be configured to communicate ultrasonically. For example, the various instances of monitoring system 200 included within monitoring network 300 may communicate ultrasonically with each other as described above with reference to FIG. 1 and FIGS. 2A and 2B.

Within monitoring network 300, different instances of monitoring system 200 may be individually identified. For example, the different instances of monitoring system 200 may each use a unique carrier frequency or frequency band. The specific instance of monitoring system 200 from which a network message originated may thus be identified by the frequency of the monitoring-network signal. In some examples, an instance of monitoring system 200 may utilize its assigned carrier frequency or frequency band for its own originated network communications, while using the carrier frequency or frequency band of a received network signal when rebroadcasting a network communication originating from another instance of monitoring system 200.

In various examples, unique addresses may be digitally encoded into network communications to identify the originating instance of monitoring system 200. When using a digitally-encoded address to distinguish different instances of monitoring system 200, each monitoring system 200 may utilize the same or different carrier frequency or frequency band. Due to the small number of monitoring systems 200 (such as smoke detectors) employed in a building, the digitally encoded address may utilize a minimal number of address bits to encode, which may allow for very brief transmission times and thereby further save energy consumption.

In various examples, the carrier frequencies used by the different instances of monitoring system may be tuned in real-time to improve signal quality and the strength of reception. For example, monitoring network 300 may perform a frequency sweep whereby network communications are sent between different instances of monitoring system 200 at different frequencies. In some examples, the respective control circuits 106 of two or more of the plurality of monitoring systems 200 may be configured to perform a frequency sweep across multiple ultrasonic carrier frequencies to test a signal strength of communications therebetween at the multiple ultrasonic carrier frequencies, and to select an ultrasonic carrier frequency for a transmission of the monitoring-network signal based on the results of the frequency sweep. For example, the carrier frequency or frequency band utilized by each monitoring system 200 may be selected based on the monitored signal strengths. In some examples, the selected carrier frequency may be the same across different instances of monitoring system 200. In other examples, the selected carrier frequency may be different across different instances of monitoring system 200.

FIG. 4 illustrates a monitoring network 400 in accordance with various examples of the present disclosure. Monitoring network 400 may be implemented in any suitable fashion according to the operation described in the present disclosure. As shown in FIG. 4, monitoring network 400 may include a plurality of monitoring systems, such as monitoring system 200. The various instances of monitoring system 200 included within monitoring network 400 may communicate with each other as described above with reference to FIG. 1, FIG. 2A, FIG. 2B, and FIG. 3.

As shown in FIG. 4, monitoring network 400 may include listening station 410. Listening station 410 may be implemented in any suitable fashion according to the operation described in the present disclosure. In various examples, listening station 410 may include one or more microphones 108 configured to receive network communications via ultrasonic acoustic waves from one or more instances of monitoring system 200. In some examples, one or more listening stations 410 may be installed in a monitoring network such as monitoring network 400 to allow for wireless listening. For example, listening station 410 may listen for and receive network communications from one or more instances of monitoring system 200. In some examples, listening station 410 may be communicatively coupled to a central panel and configured to message the central panel in response to receiving a monitoring-network signal from one of the plurality of monitoring systems 200. For example, listening station 410 may include a wired communication link or a wireless communication link (for example, cellular or Wi-Fi) to a central panel or a central server. Listening station 410 may thus relay network communications from one or more instances of monitoring system 200 to a remote central panel or remote central server out of range from the monitoring systems 200.

In some examples, monitoring network 400 may include multiple instances of listening station 410. In such examples, the different instances of listening station 410 may be placed at different locations within a residential, commercial, or industrial building, and may thus collectively implement three-dimensional listening to determine a location of the monitoring system 200 initiating a network communication such as a status update, a detected environmental condition, or a detected hazard.

As described above with reference to FIG. 3, each monitoring system 200 may utilize a different carrier frequency or frequency band and may thus be individually identified by the carrier frequency or frequency band of a received signal. In such examples, listening station 410 may be configured to listen specifically to the relevant frequencies for one or more instances of monitoring system 200 that are within range of the listening station 410.

In various examples, the carrier frequencies used by the different instances of monitoring system 200 in monitoring network 400 may be tuned in real-time to improve signal quality and the strength of reception at listening station 410. For example, monitoring network 400 may perform a frequency sweep whereby network communications are transmitted by instances of monitoring system 200 at different frequencies. In some examples, listening station 410 and the respective control circuits 106 of the plurality of monitoring systems 200 may be configured to perform a frequency sweep across multiple ultrasonic carrier frequencies to test a signal strength of communications therebetween at the multiple ultrasonic carrier frequencies, and to select an ultrasonic carrier frequency for a transmission of the monitoring-network signal based on the results of the frequency sweep. The signal strength for each transmission may be monitored. The carrier frequency or frequency band utilized by each monitoring system 200 may then be selected based on the strengths of the signals received at listening station 410 or at another instance of monitoring system 200. In some examples, the selected carrier frequency may be the same across different instances of monitoring system 200. In other examples, the selected carrier frequency may be different across different instances of monitoring system 200.

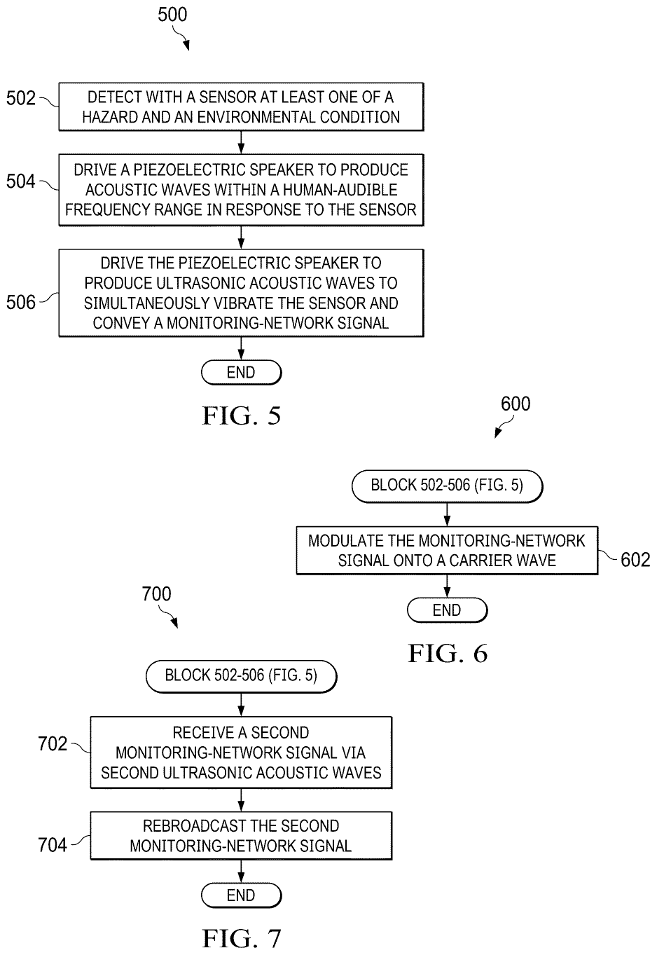

FIG. 5 illustrates a flow chart of an example method 500 for operating a monitoring system in accordance with various examples of the present disclosure. According to one example, method 500 may begin at block 502. Teachings of the present disclosure may be implemented in a variety of configurations of monitoring system 100 and monitoring system 200. As such, the initialization point for method 500 and the order of 502-506 comprising method 500 may depend on the implementation chosen.

At block 502, at least one of a hazard and an environmental condition may be detected with a sensor. At block 504, a piezoelectric speaker may be driven to produce acoustic waves within a human-audible frequency range in response to the sensor. At block 506, the piezoelectric speaker may be driven to produce ultrasonic acoustic waves to simultaneously vibrate the sensor and convey a monitoring-network signal.

Although FIG. 5 discloses a particular number of operations related to method 500, method 500 may be executed with greater or fewer operations than those depicted in FIG. 5. For example, after block 506, method 500 may continue with the additional operations illustrated in FIG. 6, the additional operations illustrated in FIG. 7, or the additional operations illustrated in both FIG. 6 and FIG. 7. In addition, although FIG. 5 discloses a certain order of operations to be taken with respect to method 500, the operations comprising method 500 may be completed in any suitable order.

FIG. 6 illustrates a flow chart of an example method 600 for operating a monitoring system in accordance with various examples of the present disclosure. Teachings of the present disclosure may be implemented in a variety of configurations of monitoring system 100 and monitoring system 200. As such, the initialization point for method 600 may depend on the implementation chosen.

Method 600 may begin with blocks 502-506 (FIG. 5) and then proceed to block 602. At block 602, the monitoring-network signal maybe be modulated onto a carrier wave. Although FIG. 6 discloses a particular number of operations related to method 600, method 600 may be executed with greater or fewer operations than those depicted in FIG. 6. In addition, although FIG. 6 discloses a certain order of operations to be taken with respect to method 600, the operations comprising method 600 may be completed in any suitable order. For example, block 602 may be executed any time after block 502.

FIG. 7 illustrates a flow chart of an example method 700 for operating a monitoring system in accordance with various examples of the present disclosure. Teachings of the present disclosure may be implemented in a variety of configurations of monitoring system 200, monitoring network 300, monitoring network 400. As such, the initialization point for method 700 may depend on the implementation chosen.

Method 700 may begin with blocks 502-506 (FIG. 5) and then proceed to block 702. At block 702, a second monitoring-network signal may be received via second ultrasonic acoustic waves. At block 704, the second monitoring-network signal may be rebroadcast. Although FIG. 7 discloses a particular number of operations related to method 700, method 700 may be executed with greater or fewer operations than those depicted in FIG. 7. In addition, although FIG. 7 discloses a certain order of operations to be taken with respect to method 700, the operations comprising method 700 may be completed in any suitable order.

Although examples have been described above, other variations and examples may be made from this disclosure without departing from the spirit and scope of these disclosed examples.

1. A monitoring system comprising:
a sensor;
a piezoelectric speaker; and
a control circuit configured to drive the piezoelectric speaker to produce ultrasonic acoustic waves that simultaneously vibrate the sensor and convey a monitoring-network signal.

2. The monitoring system of claim 1, wherein the monitoring-network signal indicates at least one of a status of the monitoring system, a detected hazard, and a detected environmental condition.

3. The monitoring system of claim 1, wherein the ultrasonic acoustic waves are in a frequency range greater than 20 kHz and less than or equal to 250 kHz.

4. The monitoring system of claim 1, wherein the control circuit is further configured to drive the piezoelectric speaker to produce acoustic waves within a human-audible frequency range in response to the sensor.

5. The monitoring system of claim 4, wherein the piezoelectric speaker has a resonant frequency within the human-audible frequency range.

6. The monitoring system of claim 1, wherein the piezoelectric speaker is directionally aimed at the sensor.

7. The monitoring system of claim 1, further comprising a microphone configured to receive a second monitoring-network signal via second ultrasonic acoustic waves.

8. The monitoring system of claim 7, wherein the control circuit is configured to drive the piezoelectric speaker to rebroadcast the second monitoring-network signal received by the microphone.

9. The monitoring system of claim 1, wherein the control circuit comprises a modulator configured to modulate the monitoring-network signal onto a carrier wave.

10. The monitoring system of claim 1, wherein the monitoring-network signal includes a digitally-encoded address identifying the monitoring system.

11. The monitoring system of claim 1, wherein the sensor is configured to detect at least one of smoke and carbon monoxide.

12. A monitoring network comprising:
a plurality of monitoring systems configured to communicate ultrasonically, wherein each monitoring system includes:
a sensor;
a piezoelectric speaker; and
a control circuit configured to drive the piezoelectric speaker to produce ultrasonic acoustic waves that simultaneously vibrate the sensor and convey a monitoring-network signal.

13. The monitoring network of claim 12, wherein:
each monitoring system further includes a microphone configured to receive a second monitoring-network signal from another of the plurality of monitoring systems; and
the control circuit of each monitoring system is configured to drive the piezoelectric speaker to rebroadcast the second monitoring-network signal.

14. The monitoring network of claim 12, further comprising a listening station including one or more microphones to receive the monitoring-network signal.

15. The monitoring network of claim 14, wherein the listening station is communicatively coupled to a central panel and configured to message the central panel in response to receiving the monitoring-network signal from one of the plurality of monitoring systems.

16. The monitoring network of claim 12, wherein respective control circuits of two or more of the plurality of monitoring systems are configured to:
perform a frequency sweep across multiple ultrasonic carrier frequencies to test a signal strength of communications therebetween at the multiple ultrasonic carrier frequencies; and
select an ultrasonic carrier frequency for a transmission of the monitoring-network signal.

17. A method for operating a monitoring system comprising:
detecting with a sensor at least one of a hazard and an environmental condition;
driving a piezoelectric speaker to produce acoustic waves within a human-audible frequency range in response to the sensor; and
driving the piezoelectric speaker to produce ultrasonic acoustic waves to simultaneously vibrate the sensor and convey a monitoring-network signal.

18. The method of claim 17, wherein the piezoelectric speaker is directionally aimed at the sensor.

19. The method of claim 17, further comprising modulating the monitoring-network signal onto a carrier wave.

20. The method of claim 17, further comprising:
receiving a second monitoring-network signal via second ultrasonic acoustic waves; and
rebroadcasting the second monitoring-network signal.
